SEQUENCE LISTING (27 sequences)

SEQ ID NO: 1 - protein, 23 aa, Homo sapiens
ENEPDLAQCFFCFKELEGWEPDD

SEQ ID NO: 2 - protein, 23 aa, Artificial Sequence
  variant of SEQ ID NO1 with a single amino acid change
ENEPDLAQMFFCFKELEGWEPDD

SEQ ID NO: 3 - protein, 6 aa, Artificial Sequence
  a six amino acid long fragment of SEQ ID NO2.
QMFFCF

SEQ ID NO: 4 - protein, 15 aa, Artificial Sequence
  a fifteen amino acid long fragment of SEQ ID NO2 and comprises the sequence of SEQ ID NO3.
DLAQMFFCFKELEGW

SEQ ID NO: 5 - protein, 10 aa, Artificial Sequence
  a ten amino acid long fragment of SEQ ID NO2 and comprises the sequence of SEQ ID NO3.
AQMFFCFKEL

SEQ ID NO: 6 - protein, 9 aa, Artificial Sequence
  a nine amino acid long fragment of SEQ ID NO2 and comprises the sequence of SEQ ID NO 3.
QMFFCFKEL

SEQ ID NO: 7 - protein, 5 aa, Artificial Sequence
  an amino acid sequence for VH CDR1 for mAb 2C2E7.
TYGMS

SEQ ID NO: 8 - protein, 17 aa, Artificial Sequence
  an amino acid sequence for VH CDR2 for mAb 2C2E7.
WINPYSGVPTYAVDFKG

SEQ ID NO: 9 - protein, 9 aa, Artificial Sequence
  an amino acid sequence for VH CDR3 for mAb 2C2E7.
GGRRGDFGY

SEQ ID NO: 10 - protein, 10 aa, Artificial Sequence
  an amino acid sequence for VL CDR1 for mAb 2C2E7.
SASSSISYMH

SEQ ID NO: 11 - protein, 7 aa, Artificial Sequence
  an amino acid sequence for VL CDR2 for mAb 2C2E7.
DTSKLAS

SEQ ID NO: 12 - protein, 8 aa, Artificial Sequence
  an amino acid sequence for VL CDR3 for mAb 2C2E7.
HQRSSHHT

SEQ ID NO: 13 - protein, 5 aa, Artificial Sequence
  an amino acid sequence for VH CDR1 for mAb 30H3D2.
SYGMS

SEQ ID NO: 14 - protein, 17 aa, Artificial Sequence
  an amino acid sequence for VH CDR2 for mAb 30H3D2.
TISSGGSHTYYPDSVRG

SEQ ID NO: 15 - protein, 14 aa, Artificial Sequence
  an amino acid sequence for VH CDR3 for mAb 30H3D2.
HPIYYYISSYAMDY

SEQ ID NO: 16 - protein, 16 aa, Artificial Sequence
  an amino acid sequence for VL CDR1 for mAb 30H3D2.
RSSQSLVHSTGNTYLH

SEQ ID NO: 17 - protein, 7 aa, Artificial Sequence
  an amino acid sequence for VL CDR2 for mAb 30H3D2.
KVSNRFS

SEQ ID NO: 18 - protein, 9 aa, Artificial Sequence
  an amino acid sequence for VL CDR3 for mAb 30H3D2.
SQSTHVPPT

SEQ ID NO: 19 - protein, 137 aa, Artificial Sequence
  an amino acid sequence for heavy chain variable region from mAb 2C2E7.
MGWLWNLLFLMAAAQSAQAQIQLVQSGPELKKPGETVKISCKASGYTFTTYGMSWVKQAPGRGLKWMGWINPYSGVPTYAVDFKGRFAFSLETSASTAYLQINNLKNEDTATYFCARGGRRGDFGYWGQGTTLTVSS

SEQ ID NO: 20 - protein, 127 aa, Artificial Sequence
  an amino acid sequence for light chain variable region from mAb 2C2E7.
MDFQVQIFSFLLISASVILSSGQIGLTQSPAIMSASPGEKVTMTCSASSSISYMHWYQQKPGTSPKTWIYDTSKLASGVPARFSGSGSGTSYSLTISSMEAEDAATYYCHQRSSHHTFGGGTKLEIK

SEQ ID NO: 21 - protein, 142 aa, Artificial Sequence
  an amino acid sequence for heavy chain variable region from mAb 30H3D2.
MNFGLSLIFLALILKGVQCEVQLVESGGDLVKPGGSLKLSCAASGFTFSSYGMSWVRLTPDKRLEWVATISSGGSHTYYPDSVRGRFTISRDNAKNTLYLQMSSLKSEDTAMYYCARHPIYYYISSYAMDYWGQGTSVTVSS

SEQ ID NO: 22 - protein, 131 aa, Artificial Sequence
  an amino acid sequence for light chain variable region from mAb 30H3D2.
MKLPVRLLVLMFWIPASSSDVVMTQTPLSLPVSLGDQASISCRSSQSLVHSTGNTYLHWYLQKPGQSPKLLIYKVSNRFSGVPDRFGGSGSGTDFTLKISRVEAEDLGVYFCSQSTHVPPTFGGGTKLEIK

SEQ ID NO: 23 - protein, 411 aa, Artificial Sequence
  a nucleotide sequence encoding the heavy chain variable region of 2C2E7 (it encodes the amino acid sequence of SEQ ID NO19).
ATGGGTTGGCTGTGGAACTTGCTATTCCTGATGGCAGCTGCCCAAAGTGCCCAAGCACAGATCCAGTTGGTACAATCTGGACCTGAGCTGAAGAAGCCTGGAGAGACAGTCAAGATCTCCTGCAAGGCTTCTGGGTATACCTTCACAACCTATGGAATGAGCTGGGTGAAACAGGCTCCAGGAAGGGGTTTAAAGTGGATGGGCTGGATAAACCCCTACTCTGGAGTGCCAACATATGCTGTTGACTTCAAGGGACGGTTTGCCTTCTCTTTGGAAACCTCTGCCAGCACTGCCTATTTGCAGATCAACAACCTCAAAAATGAGGACACGGCTACATATTTCTGTGCAAGAGGAGGGCGGAGGGGGGACTTTGGCTACTGGGGCCAAGGCACCACTCTCACAGTCTCCTCA

SEQ ID NO: 24 - protein, 381 aa, Artificial Sequence
  a nucleotide sequence encoding the light chain variable region of 2C2E7 (it encodes the amino acid sequence of SEQ ID NO20).
ATGGATTTTCAGGTGCAGATTTTCAGCTTCCTGCTAATCAGTGCCTCAGTCATACTGTCCAGCGGACAAATTGGTCTCACCCAGTCTCCAGCAATCATGTCTGCATCTCCAGGGGAGAAGGTCACCATGACCTGCAGTGCCAGCTCAAGTATAAGTTACATGCATTGGTACCAGCAGAAGCCAGGCACCTCCCCCAAAACATGGATTTATGACACATCCAAACTGGCTTCTGGAGTCCCTGCTCGCTTCAGTGGCAGTGGGTCTGGGACCTCTTATTCTCTCACAATCAGCAGCATGGAGGCTGAAGATGCTGCCACTTATTACTGCCATCAGCGGAGTAGTCACCACACGTTCGGAGGGGGGACCAAGCTGGAAATAAAA

SEQ ID NO: 25 - protein, 426 aa, Artificial Sequence
  a nucleotide sequence encoding the heavy chain variable region of 30H3D2 (it encodes the amino acid sequence of SEQ ID NO21).
ATGAACTTCGGGCTCAGCTTGATTTTCCTTGCCCTTATTTTAAAAGGTGTCCAGTGTGAGGTGCAGCTGGTGGAGTCTGGGGGAGACTTAGTGAAGCCTGGAGGGTCCCTGAAACTCTCCTGTGCAGCCTCTGGATTCACTTTCAGTAGCTATGGCATGTCTTGGGTTCGCCTGACTCCAGACAAGAGGCTGGAGTGGGTCGCAACCATTAGCAGTGGTGGTAGTCACACCTACTATCCAGACAGTGTGAGGGGGCGATTCACCATCTCCAGAGACAATGCCAAGAACACCCTGTACCTGCAAATGAGCAGTCTGAAGTCTGAGGACACAGCCATGTATTACTGTGCAAGACACCCAATTTATTACTACATTAGTAGCTATGCTATGGACTACTGGGGTCAAGGAACCTCAGTCACCGTCTCCTCA

SEQ ID NO: 26 - protein, 393 aa, Artificial Sequence
  a nucleotide sequence encoding the light chain variable region of 30H3D2 (it encodes the amino acid sequence of SEQ ID NO22).
ATGAAGTTGCCTGTTAGGCTGTTGGTGCTGATGTTCTGGATTCCTGCTTCCAGCAGTGATGTTGTGATGACCCAAACTCCACTCTCCCTGCCTGTCAGTCTTGGAGATCAAGCCTCCATCTCTTGCAGATCTAGTCAGAGCCTTGTACACAGTACTGGAAACACCTATTTACATTGGTACCTGCAGAAGCCAGGCCAGTCTCCAAAGCTCCTGATCTACAAAGTTTCCAACCGATTTTCTGGGGTCCCAGACAGGTTCGGTGGCAGTGGATCAGGGACAGATTTCACACTCAAGATCAGCAGAGTGGAGGCTGAGGATCTGGGAGTTTATTTCTGCTCTCAAAGTACACATGTTCCTCCGACGTTCGGTGGAGGCACCAAGCTGGAAATCAAA

SEQ ID NO: 27 - protein, 15 aa, Artificial Sequence
  a fifteen amino acid long fragment of human survivin.
DLAQCFFCFKELEGW

(19) United States
(12) Patent Application Publication
(10) Pub. No.: US 2024/0317844 A1
(43) Pub. Date: Sep. 26, 2024

(54) ANTI-SURVIVIN ANTIBODIES FOR TREATMENT OF AUTOIMMUNE DISEASES

(71) Applicant: Health Research, Inc., Buffalo, NY (US)

(72) Inventors: Robert A. FENSTERMAKER, Clarence, NY (US); Michael J. CIESIELSKI, Orchard Park, NY (US)

(21) Appl. No.: 18/259,763

(22) PCT Filed: Dec. 23, 2021
(86) PCT No.: PCT/US2021/065136

§ 371 Date: Jun. 28, 2023

Related U.S. Application Data
(60) Provisional application No. 63/132,964, filed on Dec. 31, 2020.

(51) Int. Cl. C07K 16/18; A61K 39/00; A61P 37/06

(52) U.S. Cl. CPC C07K 16/18; A61P 37/06; A61K 2039/505; C07K 2317/34

(57) ABSTRACT

Provided are methods for treatment of survivin-positive autoimmune diseases comprising administration of survivin specific antibodies to subjects who are afflicted with an autoimmune disease.

### CROSS REFERENCE TO RELATED APPLICATION

This application claims priority to U.S. provisional patent application No. 63/132,964, filed Dec. 31, 2020, the entire disclosure of which is incorporated herein by reference.

### SEQUENCE LISTING

The instant application contains a Sequence Listing which has been submitted electronically in ASCII format and is hereby incorporated by reference in its entirety. Said ASCII copy was created on Dec. 22, 2021, has the title “003551_01011_Sequence_Listing_ST25.txt” and has a file size of 24,942 bytes.

### BACKGROUND OF THE DISCLOSURE

Survivin is an intracellular protein that belongs to a family of apoptosis inhibitors. Survivin acts in concert with the mitotic spindle apparatus to regulate cell division. It is expressed in certain cells during the G2/M phase of the cell cycle and associates with the spindle microtubule organizing center during this phase of cell cycle progression. Survivin functions in critical roles at a number of different cellular loci to regulate the cell cycle and to inhibit apoptotic cell death. It is frequently expressed by cancer cells of many different types, but uncommonly by normal adult tissues.

Survivin is a member of the Inhibitor of apoptosis proteins (IAPs) family and has emerged as a driver of pathology in several autoimmune disorders (Gravina, Wasen et al. 2017, Autoimmun Rev 16(8): 845-855). Studies assessing patients with primary progressive multiple sclerosis found expression of survivin in resting T cells (Hebb, Moore et al. 2008, Mult Scler 14(5): 577-594). Furthermore, survivin was over-expressed in mitogen stimulated T lymphocytes from patients with active multiple sclerosis compared to patients with stable disease and the expression correlates with disease activity (Sharief, Noori et al. 2002, Eur J Neurol 9(5): 503-510) and the expression can be reduced with interferon-beta treatment ((Sharief and Semra 2002, Arch Neurol 59(7): 1115-1121). Several investigations in rheumatoid arthritis have demonstrated survivin to maintain autoreactive T cells and support pathological synovial cells (Zafari, Rafiei et al. 2019, J Cell Physiol 234(12): 21575-21587).

Survivin peptide sequences have been used to develop vaccination strategies for cancer (Kusner, Ciesielski et al. 2014, PLOS One 9(7): e102231). However, challenges remain for effective treatment of individuals with autoimmune disorders. As such, there continues to be a need to develop additional strategies to combat autoimmune diseases. The present disclosure is related to this need.

### SUMMARY OF THE DISCLOSURE

The present disclosure provides compositions and methods for treatment of autoimmune diseases that involve survivin-expressing cells. This disclosure demonstrates the role of survivin in myasthenia gravis (MG). This disclosure provides an analysis of peripheral blood mononuclear cells (PBMCs) obtained from patients with MG and healthy controls. We confirmed the differential expression of survivin in CD20+ lymphocytes from MG patients compared to healthy controls. Furthermore, the CD20+ population of cells from MG patients contained a higher percentage of extracellular survivin compared to healthy controls. In clinically relevant mouse model of MG, an antibody directed against survivin reduced disease severity, the expression of Acetylcholine receptor (AChR)-specific antibodies, and reduced CD19+survivin+splenocytes. The present disclosure is, at least in part, based on the ability to target survivin through antibody recognition of autoreactive cells and provides a method for a highly specific therapeutic for MG. It is expected that the described antibodies will be beneficial for prophylaxis or therapy of other autoimmune disorders that involve surviving expressing cells.

The method of treatment comprises administering surviving-binding antibodies or survivin-binding fragments or variants thereof to an individual in need of treatment. The antibodies include isolated antibodies, including monoclonal and polyclonal antibodies and fragments and variants thereof, compositions comprising the antibodies, nucleic acid molecules encoding the antibodies or portions thereof or variants thereof. In embodiments, the disclosure also provides vectors encoding the antibodies, cells comprising the antibodies or recombinant polynucleotides encoding the antibodies, and kits comprising one or more antibodies or expression vectors encoding the antibodies. The disclosure provides methods of using the antibodies or nucleic acid molecules encoding them to reduce or prevent development of one or more symptoms associated with autoimmune disease.

Antibodies useful in the present methods include an isolated antibody, which may be a monoclonal antibody (mAb), which is specifically reactive against one or more epitopes of survivin. The antibody may be generated in response to administration of a peptide of survivin or a modification thereof. For example, an antibody can be generated in response to a peptide, which may be 9 to 23 amino acids long and comprises the core sequence QMFFCF (SEQ ID NO:3).

In embodiments, an antibody of the disclosure is as described in U.S. Pat. No. 10,167,340, from which the description of antibodies, antibody and antibody segment amino acid sequences, and nucleotide sequences, is incorporated herein by reference.

An antibody of this disclosure can be a monoclonal antibody comprising a heavy chain variable region (VH) comprising a complementarity-determining region (CDR) 1 having a sequence set forth in SEQ ID NO:7, a CDR2 having a sequence set forth in SEQ ID NO:8 and a CDR3 having a sequence set forth in SEQ ID NO:9, and a light chain variable region (VL) comprising a CDR1 having a sequence set forth in SEQ ID NO: 10, a CDR2 having a sequence set forth in SEQ ID NO: 11 and a CDR3 having a sequence set forth in SEQ ID NO:12; or a monoclonal antibody comprising a VH comprising a CDR1 having a sequence set forth in SEQ ID NO: 13, a CDR2 having a sequence set forth in SEQ ID NO:14 and a CDR3 having a sequence set forth in SEQ ID NO: 5, and a VL comprising a CDR1 having a sequence set forth in SEQ ID NO: 16, a CDR2 having a sequence set forth in SEQ ID NO:17 and a CDR3 having a sequence set forth in SEQ ID NO:18.

The antibodies of the present disclosure may be chimeric, human, or humanized antibodies. In chimeric or humanized antibodies, some portions of the heavy and/or light chains may be identical or homologous to sequences from one species while other portions may be identical or homologous to sequences from a different species. For example, murine monoclonal antibodies may be isolated or generated and then portions of these antibodies (or sequence information derived therefrom) used for generating chimeric or humanized antibodies. For example, mice may be immunized with one or more survivin peptides and then samples can be collected. The samples can be screened and selected to develop a panel of monoclonal antibodies and corresponding hybridoma cell lines. Portions or sequences from the monoclonal antibodies can then be used to generate chimeric or humanized antibodies. An antibody of the present disclosure can also be an antibody fragment that binds with specificity to a survivin epitope, a single chain antibody, a bispecific antibody, or tri-specific antibody, which binds with specificity to a surviving epitope.

The disclosure provides nucleic acid molecules comprising sequences encoding portions or all of the antibodies (including mAbs) sequences. The disclosure also provides cells comprising the nucleic acid molecules.

This disclosure provides a method of treating an individual afflicted with an autoimmune disease or at risk of having an autoimmune disease comprising administering to the individual a composition comprising one or more antibodies that are specific for survivin, such as, for example, human survivin.

In an embodiment, this disclosure provides a method for treatment of survivin-positive autoimmune disease comprising administering to the individual a composition comprising one or more antibodies that are specific for survivin, such as, for example, human survivin. In an embodiment, this disclosure provides a method for treatment of survivin positive autoimmune disease (e.g., MG) an antibody comprising a heavy chain variable region (VH) comprising a complementarity-determining region (CDR) 1 having a sequence set forth in SEQ ID NO:7, a CDR2 having a sequence set forth in SEQ ID NO:8 and a CDR3 having a sequence set forth in SEQ ID NO:9, and a light chain variable region (VL) comprising a CDR 1 having a sequence set forth in SEQ ID NO:10, a CDR2 having a sequence set forth in SEQ ID NO:11 and a CDR3 having a sequence set forth in SEQ ID NO: 12; or a monoclonal antibody comprising a VH comprising a CDR1 having a sequence set forth in SEQ ID NO:13, a CDR2 having a sequence set forth in SEQ ID NO:14 and a CDR3 having a sequence set forth in SEQ ID NO: 5, and a VL comprising a CDR1 having a sequence set forth in SEQ ID NO:16, a CDR2 having a sequence set forth in SEQ ID NO:17 and a CDR3 having a sequence set forth in SEQ ID NO:18.

### BRIEF DESCRIPTION OF THE FIGURES

FIG. 1. Intracellular and extracellular localization of survivin+in CD20+PBMCs from MG patients. MG patients and healthy controls were assessed for survivin expression by FACS analysis. Samples were viewed on a BD Celesta analyzer and survivin+CD20+ cells were determined by FlowJo. (A) Representative dot plot from one PBMC sample. The area to determine percent positive CD20 and survivin cells is shown. (B) The graph depicts the percent of CD20+ cells that were positive for intracellular survivin staining in MG patients (n=29) and healthy controls (n=15). (C) The graph of extracellular localization of survivin in CD20+ cells from MG patients (n=29) and healthy controls (n=15). Student's t-test was used to determine statistical significance.

FIG. 2. Mouse model of EAMG demonstrated efficacy of treatment with antibody to survivin. Mice (n=27) were injected with AChR in CFA followed by injections with AChR in IFA twice (day 28 and 52). Treatment was initiated on day 54 with i.p. injections of high dose anti-survivin (100 mcg), low dose anti-survivin (20 mcg) or PBS. Treatment was given every other day for a total of six injections. Termination of the experiment occurred on day 67 with final weight (A), final disease score (B), grip strength over the course of the experiment (C), and final grip strength (D) noted. Student's t-test was used to confirm statistical relevance.

FIG. 3. Splenocytes from anti-survivin treated EAMG animals demonstrated lower percentage of CD19+survivin+ cells. Splenocytes were excised from the EAMG and control animal on day 67. Cells were stained for viability dye, CD3, CD4, and CD19. Cells were permeabilized and stained for survivin. Samples were viewed on a BD Celesta analyzer and survivin+CD19+ cells were determined by FlowJo. (A) Representative dot plot from PBS-CFA (control), and EAMG animals (PBS treated, high dose anti-survivin, and low dose anti-survivin) are shown. (B) Scatterplot of all samples analyzed for survivin+CD19+ cells. Statistical analysis was done by Student's t-test.

FIG. 4. Antibody levels to AChR was reduced in anti-survivin treated EAMG animals. Blood was taken from the EAMG animals at time of termination. Serum was collected and assayed by ELISA for tAChR-specific total IgG (A), IgG1 (B), IgG2a (C), and IgG2b (D). Student's t-test was used to determine statistical value.

FIG. 5. Antibodies that target the NMJ were reduced in high dose anti-survivin treated EAMG animals. TA muscle was sectioned and stained for mouse-specific IgG (A) and bungarotoxin which labeled AChR (B). Images were taken on a Carl Zeiss Cell Observer Spinning Disk Confocal Microscope and analyzed for fluorescent intensity by Zen software. Statistical comparisons were performed by Student's t-test analysis.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all steps and compositions of matter described herein in the text and figures of this disclosure, including all such steps individually and in all combinations thereof, and includes all compositions of matter. The disclosure includes all polynucleotide sequences, their RNA or DNA equivalents, all complementary sequences, and all reverse complementary sequences. If reference to a database entry is made for a sequence, the sequence is incorporated herein by reference as it exists in the database as of the effective filing date of this application or patent. Sequences that are 80.0-99.9% identical, inclusive, and including all numbers to the first decimal point there between, to any nucleotide and amino acid sequence are encompassed by this disclosure.

The singular forms “a”, “an” and “the” include plural referents unless the context clearly dictates otherwise. The terms “a” (or “an”), as well as the terms “one or more,” and “at least one” can be used interchangeably herein. Furthermore, “and/or” where used herein is to be taken as specific disclosure of each of the two specified features or components with or without the other.

Survivin is considered to be an intracellular protein that is thought not to be secreted by cells or displayed on the cell surface, except within the context of MHC class I presentation. As such, antibody-mediated (passive) survivin immunotherapy would not be expected to be effective for treating autoimmune diseases. However, that present disclosure demonstrates that passive surviving-based immunotherapy is effective for treatment of autoimmune diseases.

This disclosure provides isolated antibodies and fragments thereof, isolated nucleic acid molecules encoding antibodies or fragments thereof, cells producing antibodies or fragments thereof, vectors or cells comprising nucleic acids encoding antibodies or fragments thereof, compositions comprising any of the foregoing, methods of making any of the foregoing, and methods of using the antibodies and fragments thereof, or nucleic acid molecules in the treatment of autoimmune disorders that involve survivin-expressing cells.

A description of the sequence listings with this application is as follows:
SEQ ID NO:1 is an amino acid sequence representing a 23 amino acid long fragment of human survivin.
SEQ ID NO:2 is a variant of SEQ ID NO: 1 with a single amino acid change.
SEQ ID NO:3 is a six amino acid long fragment of SEQ ID NO:2.
SEQ ID NO:4 is a fifteen amino acid long fragment of SEQ ID NO:2 and comprises the sequence of SEQ ID NO:3.
SEQ ID NO:5 is a ten amino acid long fragment of SEQ ID NO:2 and comprises the sequence of SEQ ID NO:3.
SEQ ID NO:6 is a nine amino acid long fragment of SEQ ID NO:2 and comprises the sequence of SEQ ID NO: 3.
SEQ ID NO: 7 is an amino acid sequence for VH CDR1 for mAb 2C2E7.
SEQ ID NO:8 is an amino acid sequence for VH CDR2 for mAb 2C2E7.
SEQ ID NO:9 is an amino acid sequence for VH CDR3 for mAb 2C2E7.
SEQ ID NO: 10 is an amino acid sequence for VL CDR1 for mAb 2C2E7.
SEQ ID NO:11 is an amino acid sequence for VL CDR2 for mAb 2C2E7.
SEQ ID NO: 12 is an amino acid sequence for VL CDR3 for mAb 2C2E7.
SEQ ID NO: 13 is an amino acid sequence for VH CDR1 for mAb 30H3D2.
SEQ ID NO: 14 is an amino acid sequence for VH CDR2 for mAb 30H3D2.
SEQ ID NO: 15 is an amino acid sequence for VH CDR3 for mAb 30H3D2.
SEQ ID NO: 16 is an amino acid sequence for VL CDR1 for mAb 30H3D2.
SEQ ID NO: 17 is an amino acid sequence for VL CDR2 for mAb 30H3D2.
SEQ ID NO: 18 is an amino acid sequence for VL CDR3 for mAb 30H3D2.
SEQ ID NO: 19 is an amino acid sequence for heavy chain variable region from mAb 2C2E7.
SEQ ID NO:20 is an amino acid sequence for light chain variable region from mAb 2C2E7.
SEQ ID NO:21 is an amino acid sequence for heavy chain variable region from mAb 30H3D2.
SEQ ID NO:22 is an amino acid sequence for light chain variable region from mAb 30H3D2.
SEQ ID NO:23 is a nucleotide sequence encoding the heavy chain variable region of 2C2E7 (it encodes the amino acid sequence of SEQ ID NO: 19).
SEQ ID NO:24 is a nucleotide sequence encoding the light chain variable region of 2C2E7 (it encodes the amino acid sequence of SEQ ID NO:20).
SEQ ID NO:25 is a nucleotide sequence encoding the heavy chain variable region of 30H3D2 (it encodes the amino acid sequence of SEQ ID NO:21).
SEQ ID NO:26 is a nucleotide sequence encoding the light chain variable region of 30H3D2 (it encodes the amino acid sequence of SEQ ID NO:22).
SEQ ID NO:27 is a fifteen amino acid long fragment of human survivin.

While vaccines provide one way forward in anti-survivin immunotherapy, there are several advantages of using a passive immunotherapy with antibodies. For example, a humanized monoclonal antibody: 1) would not be HLA-restricted (unlike peptide vaccines), 2) would be doseable, and 3) could be used in conjunction with a vaccine or other drugs or therapies. One or more the above advantages are also applicable to mAbs in general, including chimeric, human and humanized antibodies.

The term “survivin peptide” or “survivin peptides” as used herein means fragments of full length survivin and includes variants of the peptides which can generate antibodies that react with the wild type survivin, such as human survivin. The term “anti-survivin antibodies” as used herein means antibodies that are generated in response to survivin or one or more survivin peptides (including variants thereof).

The disclosure provides a method for treatment of survivin-positive autoimmune diseases. Survivin-positive autoimmune diseases are pathological conditions in which long-lived survivin-expressing immune cells direct an unwanted attack against normal human tissues causing disease or illness. These disease states are frequently accompanied by pathologic injury or destruction of normal tissues or organs. Examples of such autoimmune diseases include achalasia, Addison's disease, adult Still's disease, alopecia areata, amyloidosis, ankylosing spondylitis, anti-GBM/Anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS), eosinophilic granulomatosis (EGPA), Cogan's syndrome, cold agglutinin disease, congenital heart block, COVID-19-associated multisystem inflammatory syndrome in children (MIS-C), Coxsackie myocarditis, CREST syndrome, Crohn's disease, dermatitis herpetiformis, dermatomyositis, Devic's disease (neuromyelitis optica), discoid lupus, Dressler's syndrome, endometriosis, epidermolysis bullosa acquisita, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), Herpes gestationis, pemphigoid gestationis (PG), hidradenitis suppurativa (HS), hypophysitis, IgA-mediated bullous dermatosis, IgA nephropathy, IgG4-related sclerosing disease, immune thrombocytopenia purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosis, ligneous conjunctivitis, linear IgA disease (LAD), lupus, Lyme disease, Meniere's disease, microscopic polyangiitis (MPA), mixed connective tissue disease (MCTD), Mooren's ulcer, myasthenia gravis (MG), Mucha-Habermann disease, multifocal motor neuropathy (MMN), multiple sclerosis, myositis, narcolepsy, neonatal Lupus, neuromyelitis optica, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II and III, polymyalgia rheumatica, polymyositis, post-myocardial infarction syndrome, post-pericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenia purpura (TTP), thyroid eye disease (TED), Tolosa-Hunt syndrome (THS), transverse myelitis, type 1 diabetes mellitus, ulcerative colitis (UC), undifferentiated connective tissue disease (UCTD), uveitis, vasculitis, vitiligo or Vogt-Koyanagi-Harada Disease.

The method comprises administering to an individual in need of treatment, a therapeutically effective amount of an antibody of the present disclosure or a fragment thereof. In an example, the autoimmune disease may be myasthenia gravis, rheumatoid arthritis, multiple sclerosis, or any of the autoimmune diseases described above. The antibody may be administered at a concentration range from 0.1 mg/ml to 100 mg/ml, 1 mg/ml to 10 mg/ml, 1 mg/ml to 50 mg/ml, 1 mg/ml to 100 mg/ml, 10 mg/ml to 100 mg/ml, or 50 mg/ml to 100 mg/ml of each of the antibodies or total antibodies. For example, a pharmaceutical composition may be administered which comprises at least or about 0.1 mg/ml, at least or about 1 mg/ml, at least or about 5 mg/ml, at least or about 10 mg/ml, at least or about 50 mg/ml, at least or about 100 mg/ml of an antibody. The antibody compositions can be administered by any suitable route and over a suitable period of time, which may be days, weeks, months or may be in the form of a maintenance therapy over a number of years. The dosage may be adjusted based on response of the individual to the therapy.

Useful compositions for the present method comprise antibodies or fragments thereof, including human antibodies, humanized antibodies, or chimeric antibodies, which are reactive against one or more epitopes of survivin. Examples of suitable survivin epitopes or variants thereof are provided in U.S. Pat. Nos. 7,943,138, and 8,580,269, the disclosures of which are incorporated herein by reference. The compositions of the present disclosure comprise antibodies generated in response to administering a peptide that is identical to a sequence within human survivin or is a variant thereof (such as at least 95% identical). For example, antibodies may be generated in and isolated from an individual following administration of a peptide that is variant of the following portion of survivin sequence ENEPDLAQCFFCFKELEGWEPDD (SEQ ID NO:1). The variant can be ENEPDLAQMFFCFKELEGWEPDD (SEQ ID NO:2-a C to M change at position 9 of SEQ ID NO:1). The peptides administered can be from 9 to 23 (including all integers therebetween) contiguous amino acids of SEQ ID NO:2, wherein the peptide comprises the core sequence of QMFFCF (SEQ ID NO:3). Exemplary survivin peptides include DLAQMFFCFKELEGW (SEQ ID NO:4), AQMFFCFKEL (SEQ ID NO:5), and QMFFCFKEL (SEQ ID NO:6). The isolated antibodies or fragments thereof may be used without modifications, or they may be engineered, such as, for example, to generate chimeric or humanized antibodies or various fragments as described therein. In one embodiment, humanized antibodies or fragments thereof are generated that are reactive against the peptide DLAQMFFCFKELEGW (SEQ ID NO:4).

A reference to the term “antibody” in this disclosure includes all its antigen-binding fragments and antigen binding variants or derivatives thereof, unless indicated otherwise. The term “Antibody” as used herein can encompass whole antibody molecules, full-length immunoglobulin molecules, such as naturally occurring full-length immunoglobulin molecules or full-length immunoglobulin molecules formed by immunoglobulin gene fragment recombinatorial processes, as well as antibody fragments. Antibody fragments can be fragments comprising at least one antibody-antigen binding site. Antibody fragments can, for example, exhibit specific binding to survivin or fragments thereof comprising the motif DLAQCFFCFKELEGW (SEQ ID NO:27). The term “antibody” includes e.g. monoclonal, multispecific (for example bispecific), recombinant, human, chimeric and humanized antibodies. The term “antibody” can also encompass recombinantly expressed antigen binding proteins and antigen binding synthetic peptides. Further, the term “antibody” can encompass minibodies, and diabodies, all of which exhibit specific binding to survivin of a fragment thereof, including but not necessarily limited to human survivin. The term “antibody”, as used herein, can also encompass immunoglobulins produced in vivo, as well as those produced in vitro, such as, for example, by a hybridoma. An antibody of the present disclosure may be modified by, for example, acetylation, formylation, amidation, phosphorylation, or polyethylene glycolation (PEGylation), as well as glycosylation. The term “an antibody” as used herein is intended to cover all antibodies disclosed herein. In embodiments, an antibody of the disclosure may thus be whole immunoglobulin molecules, or may be antigen-binding fragments thereof, including but not limited to, Fab, F(ab′), F(ab′)2, Fv, dAb, Fd, CDR fragments, single-chain antibodies (scFv), bivalent single-chain antibodies, single-chain phage antibodies, diabodies, nanobodies and the like. The fragments of the antibodies may be produced synthetically or by enzymatic or chemical cleavage of intact immunoglobulins or may be genetically engineered by recombinant DNA techniques.

In embodiments, an antibody of the disclosure comprises at least one modification of its constant region, wherein the modification increases in vivo half-life of the antibody, or alters the ability of the antibody to bind to Fc receptors, or alters the ability of the antibody to cross placenta or to cross a blood-brain barrier or to cross a blood-testes barrier, or inhibits aggregation of the antibodies, or a combination of said modifications.

Administration of survivin peptides can be used for generation of polyclonal antibodies. For example, suitable animals can be administered one or more survivin peptides and serum can be collected. Further, human anti-survivin antibody-expressing cells can be isolated from immunized animals or patients vaccinated with survivin or survivin peptides—for example, from individuals who may be participating in clinical trials. IgG+ memory B cells from patient samples can be expanded and induced to differentiate into IgG-secreting cells, which can be screened for high-affinity target (survivin peptide) binding. The cells can also be used for generation of hybridomas. Variable regions of antibody genes can be cloned from isolated cells by RT-PCR using the PIPE method (Dodev T S et al. (2014) Scientific Reports 4, 5885. doi: 10.1038/srep058853). Recombinant human, humanized or chimeric mAbs can be constructed from these molecules and can be expressed and screened in functional and binding affinity assays and for anti-tumor activity. In this regard, we have been able to detect specific antibodies by ELISA in several patients in a clinical study. Samples can be frozen for later use for isolation of memory B cells.

In one embodiment, this disclosure provides isolated antibodies. By the term “isolated” it is meant that the antibody or the fragment thereof, is separated and/or recovered from its natural environment. The isolation of the antibody from its natural environment can be such that the antibody can be used without interference from other active agents (such as other proteins) that normally are present in its natural environment.

In one embodiment, this disclosure provides generating and isolating single domain antibodies or nanobodies produced by camelids in response to introducing survivin or survivin peptides into the camelids. The nanobodies are typically heavy chain antibodies and thus contain heavy chain homodimers and do not contain antibody light chains. These antibodies typically comprise a single variable domain and two constant domains (CH2 and CH3).

The antibodies of the present disclosure may be obtained from a human or a non-human animal. In many mammals, intact immunoglobulins have two heavy chains and two light chains. Each of the light chains is covalently linked to a heavy chain by a disulfide bond. The two heavy chains are linked to each other by additional disulfide bonds. The light chain typically has one variable domain (VL) and one constant domain (CL). The heavy chain can also have one variable domain (VH). The variable domains contain complementarity-determining regions (CDRs). The heavy chain can further have three or four constant domains (CHI, CH2, CH3 and CH4). The variability of the constant domains results is various isotypes such as IgA, IgD, IgE, IgG, and IgM.

The CDRs are primarily responsible for binding to an epitope of an antigen. The CDRs of each chain are typically referred to as CDR1, CDR2, and CDR3, numbered sequentially starting from the N-terminus, and are typically identified by the chain in which the particular CDR is located. Thus, a VH CDR3 (or VH-CDR3) is located in the variable domain of the heavy chain of the antibody in which it is found, whereas a VL CDR1 (or VL-CDR1) is the CDR1 from the variable domain of the light chain of the antibody in which it is found. An antibody that binds survivin or survivin peptides, for example, will have a specific V_{H} region and the V_{L} region sequence, and thus specific CDR sequences. Antibodies with different specificities (i.e. different combining sites for different antigens) have different CDRs.

The terms V_{H} or VH as used herein refer to the variable region of an immunoglobulin heavy chain, including a heavy chain of an Fv, scFv, dsFv or Fab, and the terms V_{L} or VL refer to the variable region of an immunoglobulin light chain, including a light chain of an Fv, scFv, dsFv or Fab.

The term “monoclonal antibody” refers to an antibody produced by a single clone of B-lymphocytes or by a cell into which the light and/or heavy chain genes of a single antibody have been transfected. Monoclonal antibodies are produced by methods known to those of skill in the art, for instance by making hybrid antibody-forming cells from a fusion of myeloma cells with immune spleen cells. For example, mice (or other suitable animals) may be immunized with one or more survivin peptides and then ascites fluid samples can be collected. The samples can be screened and selected to develop a panel of monoclonal antibodies and corresponding hybridoma cell lines. Murine (or other) monoclonal antibodies may be isolated or generated and then humanized, if desired.

An antibody of the present disclosure can be an antibody of any class. For example, an antibody of the present invention can be an antibody isotype IgG1, IgG2, IgG3, IgG4, IgM, IgA, IgD or IgE. For example, the antibody can be IgG2b. The term “isotype”, as used herein, can in particular refer to the antibody class (such as e.g. IgG) that is encoded by heavy chain constant region genes. Sequences of human immunoglobulin constant regions are known in the art and are available in public databases such as National Center for Biotechnology Information (NCBI), U.S. National Library of Medicine.

The term “chimeric antibody” refers to an antibody which has framework residues from one species, such as human, and CDRs (which generally confer antigen binding) from another species, such as a murine antibody that specifically binds survivin. In a chimeric antibody, some portions of the heavy and/or light chains may be identical or homologous to sequences from a particular species while other portions may be identical or homologous to sequences from a different species. Chimeric antibodies generally exhibit decreased immunogenicity and increased stability. Techniques for cloning murine immunoglobulin variable domains known in the art-such as, for example, see Orlandi et al., Proc. Natl Acad. Sci. USA 86: 3833 (1989), and Leung et al., Hybridoma 13:469 (1994). As an example of a chimeric antibody, polynucleotides encoding the variable domains of the light chain or the heavy chain of an antibody derived from an animal (e.g., mouse, rat, or chicken) other than human can be linked to polynucleotides encoding the constant domains of the light chain or the heavy chain derived from a human antibody to produce a polynucleotide (such as DNA) encoding a chimeric antibody. Examples of chimeric antibodies include those comprising SEQ ID NOs: 19 and 20, and those comprising SEQ ID NOs:20 and 21.

A “human” antibody (also called a “fully human” antibody) is an antibody that includes human framework regions and all of the CDRs from a single or different human immunoglobulins. Thus, frameworks from one human antibody can be engineered to include CDRs from a different human antibody. Methods for producing human antibodies are known in the art—such as, for example, see Mancini et al., 2004, New Microbiol. 27:315-28; Conrad and Scheller, 2005, Comb. Chem. High Throughput Screen. 8:117-26.

A “humanized antibody” is typically a human antibody that has one or more amino acid residues imported into it (i.e., introduced into it) from a source that is non-human. For example, a humanized antibody is a recombinant protein in which the CDRs of an antibody from a species such as rodent, rabbit, dog, goat, or horse are imported into human heavy and light variable domains. The constant domains (also referred to as framework regions) of the antibody molecule are generally the same as those of a human antibody. The non-human immunoglobulin providing the CDRs can be termed as “donor” and the human immunoglobulin providing the framework can be termed as “acceptor”. For example, all the CDRs can be from the donor immunoglobulin in a humanized immunoglobulin. Constant regions need not be always present, but if they are, they can be substantially identical to human immunoglobulin constant regions, i.e., at least about 85-90%, such as about 95% or more identical. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089, and U.S. Publication No. 2010/0196266). For example, murine monoclonal antibodies may be isolated or generated and then humanized. Examples of humanized antibodies include those comprising CDRs having sequences of SEQ ID NOs: 7 through 12, and those comprising CDRs having sequences of SEQ ID NOs: 13 through 18.

Antibody fragments can be produced by enzymatic digestion. For example, papain digestion of antibodies produces two identical antigen-binding fragments, called “Fab” fragments, and a “Fc” fragment. The Fab fragment contains an entire L chain and the variable region domain of the H chain (VH), and the first constant domain of one heavy chain. Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is capable of cross-linking antigen. “Fv” is the minimum antibody fragment that contains a complete antigen-recognition and -binding site and single-chain Fv also abbreviated as “sFv” or “scFv” are antibody fragments that comprise the VH and VL antibody domains connected into a single polypeptide chain. The term “diabodies” refers to small antibody fragments prepared by constructing sFv fragments with short linkers between the VH and VL domains such that inter-chain but not intra-chain pairing of the V domains is achieved, resulting in a bivalent fragment, i.e., fragment having two antigen-binding sites. A single domain antibody (sdAb) is an antibody fragment which has a single monomeric variable antibody domain. ScAbs can be made from heavy-chain antibodies found in camelids. An antibody fragment can be a single variable region or a peptide consisting of or comprising a single CDR. A single-chain antibody has a heavy chain variable domain and a light chain variable domain linearly linked to each other via a linker. A polynucleotide (such as DNA) encoding the single-chain antibody can be produced by binding a polynucleotide encoding the heavy chain variable domain, a polynucleotide encoding the linker (typically 10-20 nucleotides), and a polynucleotide encoding the light chain variable domain, with the heavy chain variable domain and the light chain variable domain being both derived from a human antibody.

The antibodies of the present invention can be bispecific or multispecific. Bispecific antibodies (diabodies) are antibodies that have binding specificities for at least two different epitopes of an antigen, such as two different epitopes of survivin. For example, a polynucleotide (such as DNA) encoding a bispecific antibody can be produced by, for example, linking in order a polynucleotide encoding a heavy chain variable region A, a polynucleotide encoding a light chain variable region B, a polynucleotide encoding a heavy chain variable domain B, and a polynucleotide encoding a light chain variable domain A. Preferably, the heavy chain variable domain and the light chain variable domain are both derived from a human antibody.

The present disclosure provides variants of sequences set forth in SEQ ID NOs: 1 through 29. For example, variants can have at least 90%, at least 95%, at least 98% or at least 99% sequence identity to the sequences disclosed in SEQ ID NOs: 1-27.

The present disclosure provides T cells transduced to express a chimeric antigen receptor (CAR). CAR molecules of the present disclosure combine antibody-based specificity for survivin with a T cell receptor-activating intracellular domain to generate a chimeric protein that exhibits specific anti-survivin, and therefore, anti-tumor cellular immune activity. A CAR molecule can comprise one or more CDRs of the heavy or light variable regions. This disclosure further provides T cells genetically modified to stably express the CAR. T cells expressing a CAR are referred to herein as CAR T cells or CAR modified T cells. For example, T cells can be genetically modified to stably express the CAR that combines a survivin recognition domain of a specific antibody, such as a monoclonal antibody described herein, with an intracellular domain of the CD3-zeta chain into a single chimeric protein.

As an example, this disclosure provides monoclonal antibodies, which can be isolated monoclonal antibodies, which specifically bind to survivin, which can be human survivin. As an example, a mAb designated 2C2 and a mAb designated H30 (or 30H3) are provided. A subclone of mAb 2C2 used for final antibody sequencing and IgG purification was designated 2C2E7, and a subclone of mAb H30 used for final antibody sequencing and IgG purification was designated 30H3D2. An antibody comprises a heavy chain variable region and a light chain variable region. The heavy chain variable region comprises a VH CDR1, a VH CDR 2, and a VH CDR3, and the light chain variable region comprises a VL CDR1, a VL CDR2, and a VL CDR3. As an example, the VH CDR1 has an amino acid sequence TYGMS (SEQ ID NO:7), the VH CDR2 has an amino acid sequence WINPYSGVPTYAVDFKG (SEQ ID NO:8), and the VH CDR3 has an amino acid sequence GGRRGDFGY (SEQ ID NO:9); and the VL CDR1 has an amino acid sequence SASSSISYMH (SEQ ID NO:10), the VL CDR2 has an amino acid sequence DTSKLAS (SEQ ID NO:11), and the VL CDR3 has an amino acid sequence HQRSSHHT (SEQ ID NO:12). As another example, the VH CDR1 has an amino acid sequence SYGMS (SEQ ID NO:13), the VH CDR2 has an amino acid sequence TISSGGSHTYYPDSVRG (SEQ ID NO: 14), and the VH CDR3 has an amino acid sequence HPIYYYISSYAMDY (SEQ ID NO:15); and the VL CDR1 has an amino acid sequence RSSQSLVHSTGNTYLH (SEQ ID NO:16), the VL CDR2 has an amino acid sequence KVSNRFS (SEQ ID NO:17), and the VL CDR3 has an amino acid sequence SQSTHVPPT (SEQ ID NO:18).

An antibody of the present disclosure can be an antibody which has VH CDRs that have 1 or 2 amino acids that are different than the sequence set forth in SEQ ID NOs:7, 8, 9, and/or which has VL CDRs that have 1 or 2 amino acids that are different than the sequence set forth in SEQ ID NOs: 10, 11, 12. An antibody of the present disclosure can be an antibody which has VH CDRs that have 1 or 2 amino acids that are different than the sequence set forth in SEQ ID NOs: 13, 14, 15, and/or which has VL CDRs that have 1 or 2 amino acids that are different than the sequence set forth in SEQ ID NOs: 16, 17, 18.

An antibody of the present disclosure can be an antibody wherein the heavy chain variable region comprises the sequence of SEQ ID NO: 19, and the light chain variable region comprises the sequence of SEQ ID NO: 20. In the sequence of SEQ ID NO: 19, amino acids 1 through 19 represent a leader sequence, amino acids 20 through 49 represent framework region (FR) 1, amino acids 50 through 54 represent CDR1, amino acids 55 through 68 represent FR2, amino acids 69 through 85 represent CDR2, amino acids 86 through 117 represent FR3, amino acids 118 through 126 represent CDR3, and amino acids 127 through 137 represent FR4. In the sequence of SEQ ID NO: 20, amino acids 1 through 22 represent a leader sequence, amino acids 23 through 45 represent FR1, amino acids 46 through 55 represent CDR1, amino acids 56 through 70 represent FR2, amino acids 71 through 77 represent CDR2, amino acids 78 through 109 represent FR3, amino acids 110 through 117 represent CDR3, and amino acids 118 through 127 represent FR4.

An antibody of the present disclosure can be an antibody comprising a heavy chain variable region comprising the sequence of SEQ ID NO:21, and a light chain variable region comprising the sequence of SEQ ID NO:22. In the sequence of SEQ ID NO:21, amino acids 1 through 19 represent a leader sequence, amino acids 20 through 49 represent FR1, amino acids 50 through 54 represent CDR1, amino acids 55 through 68 represent FR2, amino acids 69 through 85 represent CDR2, amino acids 86 through 117 represent FR3, amino acids 118 through 131 represent CDR3, and amino acids 132 through 142 represent FR4. In the sequence of SEQ ID NO: 22, amino acids 1 through 19 represent a leader sequence, amino acids 20 through 42 represent FR1, amino acids 43 through 58 represent CDR1, amino acids 59 through 73 represent FR2, amino acids 74 through 80 represent CDR2, amino acids 81 through 112 represent FR3, amino acids 113 through 121 represent CDR3, and amino acids 122 through 131 represent FR4.

An antibody of the present disclosure can be an antibody comprising a heavy chain variable region comprising a sequence of SEQ ID NO:19 and a light chain variable region comprising a sequence of SEQ ID NO:20, or variants thereof that have 90% to 99% sequence identity. An antibody of the present disclosure can be an antibody comprising a heavy chain variable region comprising a sequence of SEQ ID NO:21 and a light chain variable region comprising a sequence of SEQ ID NO:22, or variants thereof that have 90% to 99% sequence identity. An antibody can be an antibody comprising a heavy chain variable region comprising a sequence of SEQ ID NO: 19 excluding the leader sequence (i.e., excluding amino acids 1 through 19) and/or a light chain variable region comprising a sequence of SEQ ID NO:20 excluding the leader sequence (i.e., excluding amino acids 1 through 22), or variants thereof that have 90% to 99% sequence identity. An antibody of the present disclosure can be an antibody comprising a heavy chain variable region comprising a sequence of SEQ ID NO:21 excluding the leader sequence (i.e., excluding amino acids 1 through 19) and/or a light chain variable region comprising a sequence of SEQ ID NO:22 excluding the leader sequence (i.e., excluding amino acids 1 through 19), or variants thereof that have 90% to 99% sequence identity.

An antibody of the present disclosure can be an antibody comprising a heavy chain variable region which has a sequence which is at least 90%, 91%, 92%, 93%, 94%, 95%, 96%, 97%, 98% or 99% identical to SEQ ID NO:19 and which comprises a CDR1 having a sequence of SEQ ID NO:7, a CDR2 having a sequence of SEQ ID NO:8, and a CDR3 having a sequence of SEQ ID NO:9, and/or a light chain variable region which as a sequence which is at least 90%, 91%, 92%, 93%, 94%, 95%, 96%, 97%, 98% or 99% identical to SEQ ID NO:20 and which comprises a CDR1 having a sequence of SEQ ID NO:10, a CDR2 having a sequence of SEQ ID NO:11, and a CDR3 having a sequence of SEQ ID NO:12.

An antibody of the present disclosure can be an antibody comprising a heavy chain variable region which has a sequence which is at least 90%, 91%, 92%, 93%, 94%, 95%, 96%, 97%, 98% or 99% identical to SEQ ID NO:21 and which comprises a CDR1 having a sequence of SEQ ID NO: 13, a CDR2 having a sequence of SEQ ID NO:14, and a CDR3 having a sequence of SEQ ID NO:15, and/or a light chain variable region which as a sequence which is at least 90%, 91%, 92%, 93%, 94%, 95%, 96%, 97%, 98% or 99% identical to SEQ ID NO:20 and which comprises a CDR1 having a sequence of SEQ ID NO:16, a CDR2 having a sequence of SEQ ID NO:17, and a CDR3 having a sequence of SEQ ID NO:18.

An antibody of the present disclosure can be a chimeric, human or humanized antibody comprising a heavy chain variable region comprising CDR1, CDR2 and CDR3 having the sequences of SEQ ID NOs: 7, 8 and 9 respectively, and a light chain variable region comprising CDR1, CDR2, and CDR3 having the sequences of SEQ ID NOs: 10, 11 and 12 respectively, or comprising a heavy chain variable region comprising CDR1, CDR2 and CDR3 having the sequences of SEQ ID NOs: 13, 14 and 15 respectively, and a light chain variable region comprising CDR1, CDR2, and CDR3 having the sequences of SEQ ID NOs: 16, 17 and 18 respectively.

Consensus amino acid sequence for the heavy and light chain variable regions from the mAb 2C2E7 are provided in SEQ ID NOs: 19 and 20 respectively. Consensus amino acid sequence for the heavy and light chain variable regions from the mAb 30H3D2 are provided in SEQ ID NOs:21 and 22 respectively. The corresponding nucleotide sequences for the amino acid sequences of SEQ ID NOs: 19, 20, 21 and 22 are provided in SEQ ID NOs:23, 24, 25 and 26 respectively.

The consensus amino acid sequence for heavy chain variable region from antibody 2C2E7 is shown below:

(SEQ ID NO: 19)
MGWLWNLLFLMAAAQSAQAQIQLVQSGPELKKPGETVKISCKASG
YTFTTYGMSWVKQAPGRGLKWMGWINPYSGVPTYAVDFKGRFAFS
LETSASTAYLQINNLKNEDTATYFCARGGRRGDFGYWGQGTTLTV
SS.

The consensus amino acid sequence for light chain variable region from antibody 2C2E7 is shown below:

(SEQ ID NO: 20)
MDFQVQIFSFLLISASVILSSGQIGLTQSPAIMSASPGEKVTMTC
SASSSISYMHWYQQKPGTSPKTWIYDTSKLASGVPARFSGSGSGT
SYSLTISSMEAEDAATYYCHQRSSHHTFGGGTKLEIK.

The consensus amino acid sequence for heavy chain variable region from antibody 30H3D2 is shown below:

(SEQ ID NO: 21)
MNFGLSLIFLALILKGVQCEVQLVESGGDLVKPGGSLKLSCAASG
FTFSSYGMSWVRLTPDKRLEWVATISSGGSHTYYPDSVRGRFTIS
RDNAKNTLYLQMSSLKSEDTAMYYCARHPIYYYISSYAMDYWGQG
TSVTVSS.

The consensus amino acid sequence for light chain variable region from antibody 30H3D2 is shown below:

(SEQ ID NO: 22)
MKLPVRLLVLMFWIPASSSDVVMTQTPLSLPVSLGDQASISCRSS
QSLVHSTGNTYLHWYLQKPGQSPKLLIYKVSNRFSGVPDRFGGSG
SGTDFTLKISRVEAEDLGVYFCSQSTHVPPTFGGGTKLEIK.

A nucleotide sequence encoding the amino acids of heavy chain variable domain set forth in SEQ ID NO:19 of mAb 2C2E7 is shown below:

(SEQ ID NO: 23)
ATGGGTTGGCTGTGGAACTTGCTATTCCTGATGGCAGCTGCCCAA
AGTGCCCAAGCACAGATCCAGTTGGTACAATCTGGACCTGAGCTG
AAGAAGCCTGGAGAGACAGTCAAGATCTCCTGCAAGGCTTCTGGG
TATACCTTCACAACCTATGGAATGAGCTGGGTGAAACAGGCTCCA
GGAAGGGGTTTAAAGTGGATGGGCTGGATAAACCCCTACTCTGGA
GTGCCAACATATGCTGTTGACTTCAAGGGACGGTTTGCCTTCTCT
TTGGAAACCTCTGCCAGCACTGCCTATTTGCAGATCAACAACCTC
AAAAATGAGGACACGGCTACATATTTCTGTGCAAGAGGAGGGCGG
AGGGGGGACTTTGGCTACTGGGGCCAAGGCACCACTCTCACAGTC
TCCTCA.

A nucleotide sequence encoding the amino acids of light chain variable domain set forth in SEQ ID NO:20 of mAb 2C2E7 is shown below:

(SEQ ID NO: 24)
ATGGATTTTCAGGTGCAGATTTTCAGCTTCCTGCTAATCAGTGCC
TCAGTCATACTGTCCAGCGGACAAATTGGTCTCACCCAGTCTCCA
GCAATCATGTCTGCATCTCCAGGGGAGAAGGTCACCATGACCTGC
AGTGCCAGCTCAAGTATAAGTTACATGCATTGGTACCAGCAGAAG
CCAGGCACCTCCCCCAAAACATGGATTTATGACACATCCAAACTG
GCTTCTGGAGTCCCTGCTCGCTTCAGTGGCAGTGGGTCTGGGACC
TCTTATTCTCTCACAATCAGCAGCATGGAGGCTGAAGATGCTGCC
ACTTATTACTGCCATCAGCGGAGTAGTCACCACACGTTCGGAGGG
GGGACCAAGCTGGAAATAAAA.

A nucleotide sequence encoding the amino acids of heavy chain variable domain set forth in SEQ ID NO:21 of mAb 30H3D2 is shown below:

(SEQ ID NO: 25)
ATGAACTTCGGGCTCAGCTTGATTTTCCTTGCCCTTATTTTAAAA
GGTGTCCAGTGTGAGGTGCAGCTGGTGGAGTCTGGGGGAGACTTA
GTGAAGCCTGGAGGGTCCCTGAAACTCTCCTGTGCAGCCTCTGGA
TTCACTTTCAGTAGCTATGGCATGTCTTGGGTTCGCCTGACTCCA
GACAAGAGGCTGGAGTGGGTCGCAACCATTAGCAGTGGTGGTAGT
CACACCTACTATCCAGACAGTGTGAGGGGGCGATTCACCATCTCC
AGAGACAATGCCAAGAACACCCTGTACCTGCAAATGAGCAGTCTG
AAGTCTGAGGACACAGCCATGTATTACTGTGCAAGACACCCAATT
TATTACTACATTAGTAGCTATGCTATGGACTACTGGGGTCAAGGA
ACCTCAGTCACCGTCTCCTCA.

A nucleotide sequence encoding the amino acids of light chain variable domain set forth in SEQ ID NO:22 of mAb 30H3D2 is shown below:

(SEQ ID NO: 26)
ATGAAGTTGCCTGTTAGGCTGTTGGTGCTGATGTTCTGGATTCCT
GCTTCCAGCAGTGATGTTGTGATGACCCAAACTCCACTCTCCCTG
CCTGTCAGTCTTGGAGATCAAGCCTCCATCTCTTGCAGATCTAGT
CAGAGCCTTGTACACAGTACTGGAAACACCTATTTACATTGGTAC
CTGCAGAAGCCAGGCCAGTCTCCAAAGCTCCTGATCTACAAAGTT
TCCAACCGATTTTCTGGGGTCCCAGACAGGTTCGGTGGCAGTGGA
TCAGGGACAGATTTCACACTCAAGATCAGCAGAGTGGAGGCTGAG
GATCTGGGAGTTTATTTCTGCTCTCAAAGTACACATGTTCCTCCG
ACGTTCGGTGGAGGCACCAAGCTGGAAATCAAA.

The present disclosure also provides isolated nucleotide sequences encoding all or portions of heavy chain variable regions for survivin specific antibodies. For example, the present disclosure provides an isolated nucleic acid molecule comprising the sequence of SEQ ID NOs: 23 or 25. An isolated nucleotide molecule of the present disclosure can encode all or portions of light chain variable regions for survivin specific antibodies. For example, the isolated nucleic acid molecule can comprise the sequence of SEQ ID NOs: 24 or 26. Variants of nucleic acid molecules can have at least 90% to at least 99% identity with the sequences of SEQ ID NOs: 23 or 25 for the heavy chain variable region or SEQ ID NOs: 24 or 26 for the light chain variable region.

The present disclosure also provides isolated nucleic acid molecules comprising or consisting of the sequence encoding one or more CDRs that recognize a survivin epitope-such as for example, sequences encoding SEQ ID NOs: 7 to 18. A nucleic acid molecule can consist of any of the sequences of SEQ ID NOs: 7 to 18, or a nucleic acid molecule can comprise one or more sequences of SEQ ID NOs: 7 to 18 and further comprise additional 1 to 50 nucleotides-generally flanking the sequences.

The disclosure provides cells comprising an expression vector or other polynucleotide sequence encoding the antibodies provided herein (including mAbs) or survivin binding fragments thereof. Nucleotide sequences encoding the mAbs or survivin binding fragments thereof can be expressed using any suitable expression vector, many of which are known in the art and/or are commercially available. A vector generally includes nucleic acid sequences, such as origin or replication that enables it to replicate in a host cell. A vector can also include selectable marker genes. Heavy and light chains can be expressed on a single expression vector, such as a plasmid or the heavy and light chains can be expressed on distinct plasmids in the same cell, after which the expressed heavy and light chains can form the conventional mAb architecture. The mAbs or survivin binding fragments thereof can be isolated and/or purified using conventional techniques, given the benefit of the present disclosure.

The isolated monoclonal antibodies or fragments thereof can be labeled, such as with enzymatic, fluorescent or radioactive tags or can be conjugated to effector molecules such as, for example, toxins.

The present disclosure provides pharmaceutical compositions comprising the antibodies or fragments thereof, and pharmaceutically suitable carrier. Suitable carriers include excipients, or stabilizers which are nontoxic to recipients at the dosages and concentrations employed, and include buffers such as acetate, Tris, phosphate, citrate, and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; and/or non-ionic surfactants such as Tween or polyethylene glycol (PEG). The pharmaceutical compositions may comprise other therapeutic agents.

Compositions of the present disclosure can comprise one type of monoclonal antibody or more than one type of monoclonal antibody. A composition of the disclosure can have one or more of an antibody or fragment or variant thereof. A composition can have a monoclonal and a polyclonal antibody. A composition can comprise one or more subtypes of antibodies. For example, a composition can comprise a mixture of IgG or IgM or a mixture of one or more of IgG1, IgG2, and IgG2b. A composition of the present disclosure can comprise an antibody as the only active ingredient, wherein the antibody may be monoclonal, polyclonal, chimeric, human, humanized or combinations thereof. By “active ingredient” is meant that the ingredient has an anti-tumor effect by inhibiting tumor growth. In embodiments, a method of the disclosure includes administering an antibody of this disclosure in combination none or more additional agents. In embodiments, the one or more additional agents promote immune tolerance. Agents that produce immune tolerance are known in the art. For example, an immunosuppressant agent may be used. In embodiments, the immunosuppressive agent may comprises an mTOR inhibitor, such as rapamycin, or a rapolog. In embodiments, the immunosuppressant comprises a calcineurin inhibitor, such as tacrolimus or cyclosporine, or a corticosteroid, such as cortisone prednisolone, prednisone, and triamcinolone.

A pharmaceutical composition of the disclosure can comprise one or more antibodies at a concentration range from 0.1 mg/ml to 100 mg/ml, 1 mg/ml to 10 mg/ml, 1 mg/ml to 50 mg/ml, 1 mg/ml to 100 mg/ml, 10 mg/ml to 100 mg/ml, or 50 mg/ml to 100 mg/ml of each of the antibodies or total antibodies. For example, a pharmaceutical composition of the disclosure can comprise at least or about 0.1 mg/ml, at least or about 1 mg/ml, at least or about 5 mg/ml, at least or about 10 mg/ml, at least or about 50 mg/ml, at least or about 100 mg/ml of an antibody.

The compositions of the present disclosure may be administered by routine methods known in the art. For example, the compositions comprising antibodies or fragments thereof may be administered via intravenous, intramuscular, intraperitoneal, intracerebrospinal, subcutaneous, intra-articular, intrasynovial, intrathecal, oral, topical, or inhalation routes, or by intracerebral or intra-spinal convection enhanced delivery or direct intratumoral injection. The antibodies may be administered parenterally directly at the target site (such as at or within lymph node). The compositions may be introduced as a single administration or as multiple administrations and may be introduced in a continuous manner over a period of time. In one embodiment, the composition may be administered daily for a period of at least 2 days such as, for example, for a period of 2-30 days (and all periods therebetween). In one embodiment, it is administered daily for 7-10 days. It may alternatively be administered at desired intervals (such as every 2, 3, 4, 5 days and the like).

It will be recognized by those of skill in the art that the form and character of the particular dosing regimen employed in the method of the invention will be dictated by the route of administration and other well-known variables, such as the size of the individual and the stage of the disease. Further, the compositions can be provided in the form of unit dosage forms for administration to an individual in need of treatment. Antibodies can be provided in a lyophilized form to be reconstituted prior to administration. The reconstitution medium can be sterile 0.9% saline solution or a suitable physiological buffer or water, or any other solution known in the art for reconstituting proteins prior to administration.

The disclosure also provides kits which can be used for administration to individuals in need of treatment. A kit, for example, can comprise one or more antibodies, which may be in a lyophilized form, optionally reconstitution media, and instructions for administration. A kit can comprise a single dose or multiple doses.

The term “treatment” refers to reduction in one or more symptoms or features associated with the presence of the particular condition being treated, e.g., an autoimmune disease. Treatment does not necessarily mean complete remission, nor does it preclude recurrence or relapses. In one embodiment, the method is a method of passive immunization.

The method of the invention can be performed in conjunction with use of survivin peptides as a vaccine. The compositions of the invention can be administered prior to, concurrently, or subsequent to other therapies.

In one aspect, the present disclosure provides compositions comprising an isolated antibody which is reactive against one or more epitopes of survivin wherein the isolated antibody or the antigen-binding fragment thereof binds to one or more epitopes of survivin. The antibody may be generated in response to administration of a peptide having the sequence ENEPDLAQMFFCFKELEGWEPDD (SEQ ID NO:2), or a fragment thereof (such as SEQ ID NO:4), wherein the fragment has from 9 to 23 (including all integers therebetween) contiguous amino acids of SEQ ID NO:2, and wherein the peptide comprises the core sequence of QMFFCF (SEQ ID NO:3). The composition may be such that the only antibody or antibodies present is/are the isolated antibody/antibodies generated in response to administration of survivin peptides. The composition may have other protein such as carrier proteins. The antibody may be a chimeric, human or a humanized antibody. The antibody may be a monoclonal or a polyclonal antibody, or a single chain, or multispecific antibody.

Reactivity of antibodies toward specific antigens can be measured by routine methods such as, for example, ELISA. Reactivity is an indication of the binding affinity. Binding affinity can also be measured by antigen/antibody dissociation rates or competition radioimmunoassays and the like. Specific binding of an antibody to an antigen means it binds the antigen with high affinity and does not specifically bind to unrelated antigens.

In one aspect, the disclosure provides a method of passive immunization comprising administering to an individual in need of treatment, a therapeutically effective amount of the composition comprising one or more antibodies generated in response to administration of a peptide having the sequence ENEPDLAQMFFCFKELEGWEPDD (SEQ ID NO:2), or a fragment thereof (such as SEQ ID NO:4), wherein the fragment has from 9 to 23 (including all integers therebetween) contiguous amino acids of SEQ ID NO:2, and wherein the peptide comprises the core sequence of QMFFCF (SEQ ID NO:3), and which antibodies have been isolated from the subject (human or non-human) they were raised in or obtained from a hybridoma supernatant, or may be engineered antibodies using sequences from the isolated antibodies.

The following example illustrates an embodiment of the disclosure and is not intended to be limiting.

### Example 1

This example describes the use of the described antibodies for the treatment of an autoimmune disease, Myasthenia gravis (MG). MG is an autoimmune disorder with the primary autoantigen being the skeletal muscle acetylcholine receptor (AChR) and serves as the prototypic antibody-mediated disorder. In this disclosure, we assessed the surface expression of survivin on CD20+ cells and the ability of an antibody directed against survivin to moderate the severity of MG in an animal model. Results described in this Example were obtained using the mAb referred to herein as 2C2E7. Similar results were obtained using the mAb referred to herein as 30H3D2.

### Materials and Methods

### Samples

Blood specimens (n=29) were collected from patients of the MG Center and neurology clinics at George Washington University (Table 1). For patients with MG, entrance criteria for participation were: 1) previous clinical diagnosis of MG; 2) age>18 years of age; 3) presence of serum acetylcholine receptor and, 4) willingness to participate and ability to provide informed consent. Exclusion criterion was limited to inability to provide informed consent. The MG Foundation of America Clinical Classification (Jaretzki, Barohn et al. 2000, Neurology 55(1): 16-23) was used to historically define the maximal disease severity and at the time of blood draw. Control subject (n=15) were recruited from neurology clinics at George Washington University. Control subject inclusion criteria were limited to willingness to participate and ability to provide informed consent. Control subject exclusion criteria were age<18 years of age, diagnosis of autoimmune diseases, and treatment with any immunotherapy in the previous 12 months. All participants provided written consent for inclusion in the study. The study was approved by the George Washington University Institutional Review Board.

### PBMC Isolation

Blood was collected in Vacutainer ACD (BD, Cat. #364606), and then were transferred to a 50 mL Ficoll-loaded LeucoSep tube (Fisher Scientific 07-000-983) which contains 15.5 mL Ficoll-Paque™ PLUS Media (GE Healthcare, Cat. #17144003). Centrifuge at RT for 30 min at 1000 g with the break off. PBMCs were isolated and washed three times with HBSS (Gibco™, Cat. #14-170-161), and then were re-suspended in ice cold cell culture IMDM (Gibco™, Cat. #31980030) which contains 10% Fetal Bovine Serum (Gibco™, Cat. #10437028) and 1% Penicillin-Streptomycin (Gibco™, Cat. #15070063) as 2.5 million per mL. 200 μL cells were transferred into 96 wells plate ‘Unstained Cells’ and place plate on ice, all other cells were kept on ice for cell viability staining.

### Flow Cytometry (FCM) Cell Viability Staining

PBMCs-IMDM suspension (25 million cells) were spun down at 4° C. 500 g for 5 min, and then were immediately re-suspended in 1 mL ice cold cell culture IMDM. 1 μL LIVE/DEAD™ Fixable Violet Dead Cell Stain Kit (ThermoFisher, Cat. #L34955) was added into PBMCs suspension and was mixed gently then incubated on ice for 30 minutes, protected from light. Cells were washed twice with 10 mL ice cold cell culture IMDM, and then were re-suspended in ice cold cell culture IMDM as 4×10⁶/mL. Cell suspension for one well (100 μL) were plated into 96 wells plate as designed wells.

### Flow Cytometry (FCM) Cell Extracellular Staining

Fc receptor on PBMCs were blocked by 5 μL Human TruStain FcX™ (BioLegend, Cat. #422302) placed for 10 minutes on ice, and then stained with antibodies CD20-FITC (ThermoFisher, Cat. #MA110136), CD4-PE (BD, Cat. #561844), CD45-PerCP (ThermoFisher, Cat. #MHCD4531), Survivin-Alexa Fluor® 700 (Novus Biologicals, Cat. #NB500-238AF700) or their isotype control antibodies Mouse IgG2a-FITC (ThermoFisher, Cat. #11-4732-81), Mouse IgG1-PE (BD, Cat. #555749), Mouse IgG1-PerCP (ThermoFisher, Cat. #MG131) and Mouse IgG2a-Alexa Fluor® 700 (Novus Biologicals, Cat. #IC003N) 30 minutes on ice. Single color staining and fluorescent minus Alexa Fluor® 700 were prepared at the same time. Fixation Buffer (100 μL) was added into each well 10 minutes on ice. Plate were spun down at 4° C. 500 g for 5 min. Washed twice by 220 μL ice cold Cell Staining Buffer, and then were re-suspended in 200 μL ice cold Cell Staining Buffer. Cell populations were evaluated by flow cytometry within 1 hour.

### Flow Cytometry (FCM) Cell Intracellular Staining

For intracellular staining of survivin expression, the extracellular stained PBMCs were washed twice by 220 μL ice cold 1X Intracellular Staining Permeabilization Wash Buffer (BioLegend, Cat. #421002), and then were re-suspended in 100 μL ice cold 1X Permeabilization/Wash Buffer. Survivin-Alexa Fluor® 700 (Novus Biologicals, Cat. #NB500-238AF700) or Mouse IgG2a-Alexa Fluor® 700 (Novus Biologicals, Cat. #IC003N) was added into designated wells 30 minutes on ice. 100 μL ice cold 1X Permeabilization/Wash Buffer was added into each well and then plate was spun down at 4° C. 500 g for 5 min. Washed twice by 220 μL ice cold 1X Permeabilization/Wash Buffer, and then were re-suspended in 200 μL ice cold Cell Staining Buffer. Cell populations were qualified by flow cytometry within 1 hour.

### EAMG Induction and Treatment

All animals were housed in The George Washington University Animal Research Facility in accordance with IACUC, AALAS, and AAALAT standards regulating housing conditions, cage cleaning procedure, air purity, humidity, temperature, feed quality, and light dark cycles. A veterinarian was available to monitor the animals during the course of the study. Animal use was approved by The George Washington University Institutional Animal Care and Use Committee (Permit No. A247), and all experimental outcomes are reported using the quality assurance guidelines set by NINDS. Twenty-seven female C57BL/6J mice were injected with Torpedo AChR (tAChR) in complete Freund's adjuvant (Sigma, Cat. #F5881) on day 0 and were boosted with tAChR in incomplete Freund's adjuvant (BD, Cat. #263910) on both day 28 and day 52. To test the therapeutic potential, we used a hybridoma IgG2a antibody to survivin (Kd ¼ 0.56 nmol/L) described herein. The mice were stratified by weight and were separated to three treatments groups (PBS, Anti-Survivin 100 μg and Anti-Survivin 20 μg per mice). Eight C57BL/6J mice, a control group, were injected with PBS-CFA on day 0, day 28 and day 52. On day 54, all mice received treatment once every two days for six total treatments. Blood, spleen, and tibialis anterior were collected for further investigation at termination on day 66.

### Splenic Preparation

Spleens were cut to small pieces with RPMI (Gibco™, Cat. #22400089) which contains 5% FBS (Gibco™, Cat. #10437028) and were pass through 40 μm Cells Strainers (Corning, Cat. #352340). RBC lysis buffer (Alfa Aesar™, Cat. #J62150AK) was added to cell pellets. After wash with 10 mL RPMI which contains 5% FBS, cells were re-suspended in freezing medium (90% FBS, 10% DMSO (Sigma, Cat. #D2650)) as 1˜2×10⁷ cells/mL/vial. Samples were stored in at −80° C. Flow cytometry of splenocytes

Frozen splenocytes were thawed in 37° C. water bath, and re-suspended in RPMI+10% FBS+1% Penicillin-Streptomycin (5,000 U/mL) (Gibco™, Cat. #15070063) (CRPMI). After one wash in cRPMI, cells were resuspended in dPBS (Gibco™, Cat. #14190144) with LIVE/DEAD™ Fixable cell viability dye (ThermoFisher, Cat. #L34955). Fc sites were blocked on the cells with purified anti-mouse CD16/32 Antibody (Biolegend, Cat. #101302). Cells were treated with antibodies to Cell surface marker panel: CD38-FITC (Biolegend, Cat. #100306), CD19-PE (Biolegend, Cat. #115508), CD4-APC (Biolegend, Cat. #100412), and their isotype control antibodies FITC (Biolegend, Cat. #400905), PE (Biolegend, Cat. #400507), APC (Biolegend, Cat. #400611) and Alexa Fluor 700R (R&D, Cat. #IC003N). Single color staining and fluorescent minus one were also prepared at the same time. Cells were washed and re-suspended in cold Cell Staining Buffer (Biolegend, Cat. #420201). Cell populations were qualified by flow cytometry within 2 hours. For cell intracellular marker panel included the above markers for 30 minutes on ice. The Fixation Buffer (Biolegend, Cat. #420801) was added to the well, and spun down at 4° C. 500 g. Antibody to Survivin-Alexa FluorR 700 and isotype control Mouse IgG2a kappa Alexa FluorR 700 were added in cells suspension in 100 μLIX Intracellular Staining Permeabilization Wash Buffer (Biolegend, Cat. #421002) for 30 minutes on ice and protected from the light. After three washes, cells were re-suspended in ice cold Cell Staining Buffer. Cell populations were qualified by flow cytometry within 2 hours.

### AChR-Specific Antibody Determination by Enzyme-Linked Immunosorbent Assay (ELISA)

ELISAs were performed to determine titers of immunoglobulin subtypes: total IgG, IgG1, IgG2a, and IgG2b, specific to tAChR. To detect antibodies to AChR, flat-bottomed 96-well Nunc-Immuno MicroWell plates (Sigma, Cat. #CLS3590) were coated overnight with tAChR. Blocking buffer (1×phosphate buffered saline (Sigma, Cat. #P5368) with 0.5% (v/v) Tween 20 (Sigma, Cat. #P1379) and 5% (w/v) bovine serum albumin (BSA; Sigma, Cat. #A3608)) was then added. To determine the tAChR-specific total IgG, IgG1, and IgG2b, serum from each animal was added in duplicate wells. The plate was incubated for 90 min, and then 100 μl of 1:1000 anti-mouse IgG-HRP conjugate (Alpha Diagnostic Intl. Inc, San Antonio, TX, Cat. #40120) was added. Tetramethylbenzidine (TMB; SeraCare Lifesciences, Milford, MA, Cat. #50-76-02 and #50-65-02) was then applied for up to 25 min and the reaction terminated with 1 M hydrochloric acid (KPL, Cat. #71826-1L). The optical density (OD) plate was read at 450 nm using a Varioskan Fluorescent and Luminescent Plate Reader (Fisher).

### Immunohistochemistry Tibialis Anterior Muscle

Ten micron cryosections of tibialis anterior were mounted onto Superfrost Plus treated slides (Fisher, Cat. #12-550-15), air dried for one hour, and fixed in cold acetone (Acros Organics, Cat. #423240025) for 10 min. Slides were air dried, washed in PBS and blocked in 5% BSA/PBS at room temperature. A conjugate of a-bungarotoxin Alexa 594 (BTX; Invitrogen, Cat. #B13423) and goat-anti-mouse IgG Alexa 488 (Invitrogen, Cat. #A11029) in 5% BSA/PBS were applied at al:500 dilution for 1 h at room temperature. After washing, coverslips were placed on the slides with Fluormount-G (Southern Biotech Associates Inc, Birmingham, AL, Cat. #010001). Sections were viewed on a Carl Zeiss Cell Observer Spinning Disk Confocal Microscope and images taken with a color CCD camera. NMJs were identified by fluorescently labeled bungarotoxin and pixel intensity measurements were determined with mean values obtained for each animal.

### Statistical Analysis

The data was analyzed for statistical significance using t-test with p<0.05 considered significant.

### Results

Survivin Localization in CD20. PBMC's from MG Patients

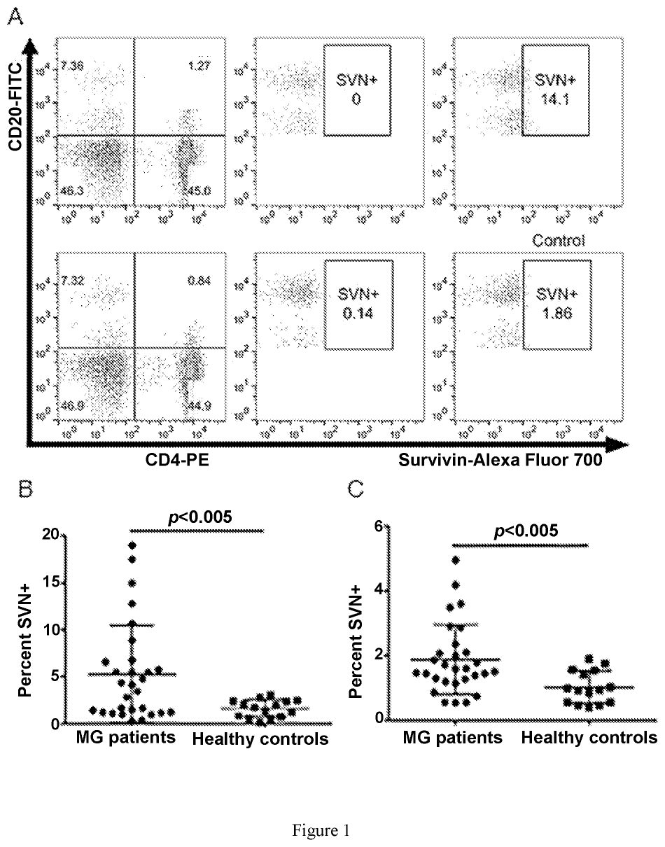

We examined the presence of survivin in CD20+ cells in PBMCs from 29 patients with MG and 15 healthy controls (FIG. 1). We found an increase percentage of survivin+CD20+ cells in MG patients compared to healthy control (5.27±5.19, 1.62±0.92, respectively, p<0.005). To examine the potential localization of survivin, we analyzed the PBMCs for cell surface staining. A greater percentage of CD20+ cells from MG patients localized survivin to the extracellular surface compared to healthy controls (1.89±1.09,1.03±0.50, respectively, p<0.005, FIG. 1C). We performed an extensive analysis of survivin expression and its relationship to disease severity, disease duration, treatment, thymic pathology, age, and gender in MG patients and healthy controls. We found no association with intracellular or extracellular thymic expression, although even among patients with long disease duration high levels of surviving expression can be found regardless of clinical severity.

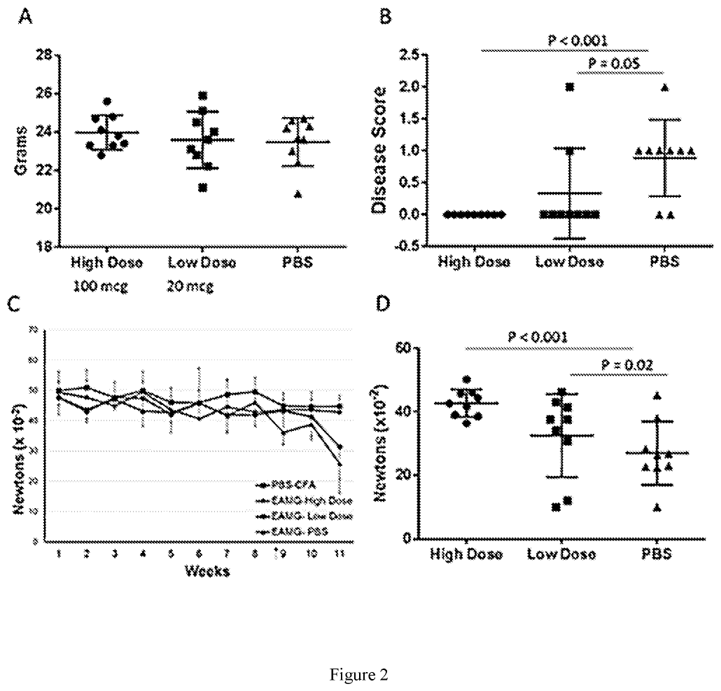

Antibody to survivin protects mice from EAMG Animals were induced with EAMG and monitored for weight and grip strength. Animals were stratified into treatment groups based on weight. Treatment with an antibody to survivin was initiated two days after the second booster of AChR followed continuing assessment in a blind fashion. Over the course of treatment, weight did not vary between groups. At end of experiment, the weights remained consistent between groups (FIG. 2A). PBS treated EAMG mice at time of sacrifice demonstrated significantly higher disease scores (FIG. 2B) compared to both the high dose (100 mg) and low dose (20 mg) anti-survivin treatment. Grip strengths were maintained in the high dose group. Whereas, the strengths were reduced in the low dose and the PBS treated groups (FIGS. 2C and D).

### Anti-Survivin Treatment Lowers Survivin Expressing B Cells in EAMG

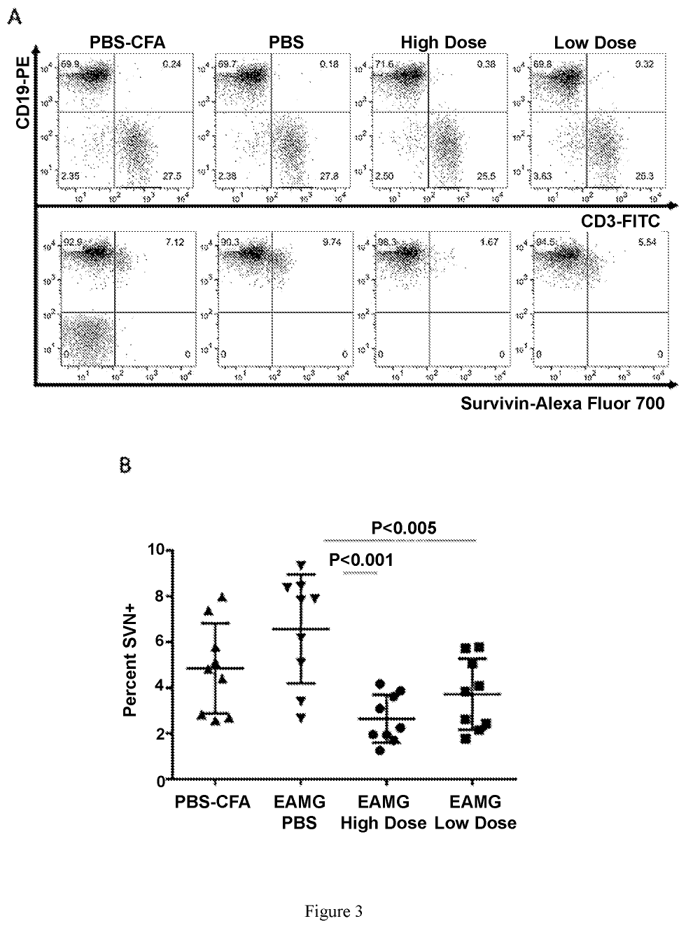

The survivin protein functions to negatively regulate apoptosis. Therefore, we postulated that immune cells in our EAMG mouse model would express survivin to maintain production of AChR receptor antibody. In our study, intracellular survivin expression was elevated in CD3-CD19+splenic B cells from EAMG animals (FIG. 3).

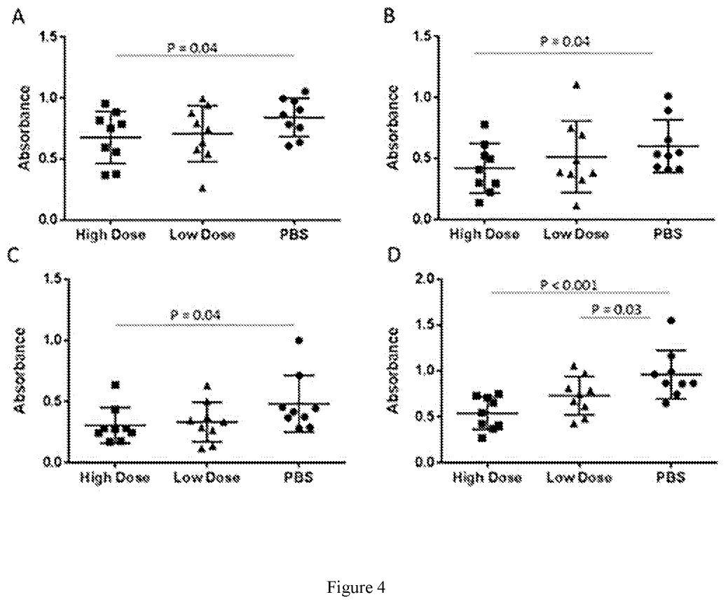

Anti-survivin treatment effectively suppressed survivin expression in a dose dependent fashion in splenic B cells (FIG. 4). The percentage of splenic CD3−/CD19+B cells expressing intracellular survivin was 4.85%±1.98% in the control CFA only group. The induction of EAMG led to a significant increase in CD3−/CD19+/SVN+B cells (6.57%±2.38%, p<0.01). The addition of antibody to survivin dramatically reduced the CD3−/CD19+/SVN+B cells in both the high dose anti-SVN group (2.65%±1.05%, p<0.000001) and the low dose anti-SVN group (3.72%±1.55%, p<0.0001). The percentage of the splenic cell population of CD3+CD19-CD4-Tc cells and CD3+CD19-CD4+Th cells and CD3-CD19+B cells did not vary among the EAMG treated groups (data not shown).

### Antibody to Survivin Lowers the Production of AChR-Specific Antibodies

All EAMG mice induced with tAChR produced detectable antibodies to the antigen. The EAMG mice treated with high dose anti-SVN demonstrated statistically lower total IgG, IgG1, IgG2a and IgG2b compared to EAMG mice treated with PBS (FIG. 4). However, EAMG mice treated with low dose anti-SVN demonstrated similar total IgG, IgG1 and IgG2a as the EAMG mice treated with PBS. The EAMG mice treated with low dose anti-SVN showed statistically lower IgG2b antibodies to tAChR compared to the PBS treated EAMG mice.

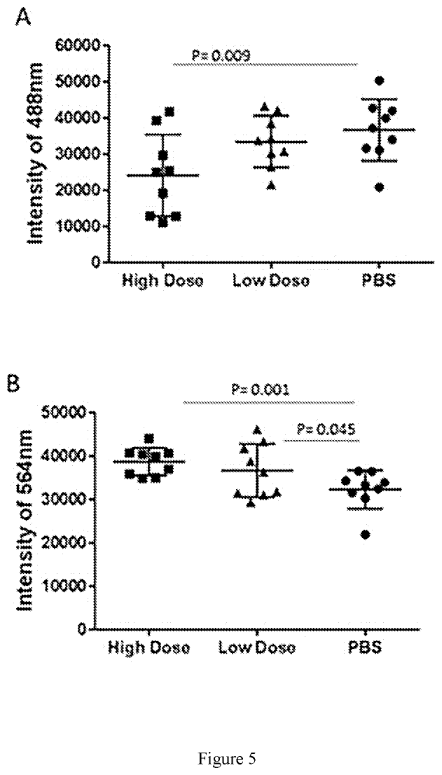

To determine the binding of the autoantibodies to the mouse NMJ and the depletion of the AChR at the cell surface, TA sections were taken from the EAMG mice treated with PBS, high dose anti-SVN and low dose anti-SVN. The binding of antibody to the AChR was significantly reduced in the high dose anti-SVN group (24205±11312 fluorescent intensity) compared to PBS treatment (36750±8504 fluorescent intensity). While, AChR expression on the cell surface of the NMJ was statistically increased in both the high and low dose anti-SVN treated EAMG mice (38785±3172, 36736±6141 fluorescent intensity, respectively) compared to the PBS treated EAMG mice (32377±4416 fluorescent intensity, FIG. 5).

### DISCUSSION

Elevated survivin expression has been found in several autoimmune diseases, although the potential mechanism and source of survivin is not clear. Here we analyzed the CD20+ cells for both intracellular and extracellular expression of survivin. Intracellular staining for survivin was found in an increased number of CD20+ cells compared to controls. We also saw an increased percentage of cells positive for extracellular expression of survivin in CD20+ cells from MG patients compared with controls. Since the expression on the extracellular surface suggests a potential therapeutic target, we assess the efficacy of an antibody to survivin to moderate weakness in a mouse model of EAMG. Post treatment of anti-survivin demonstrated improvement in disease score, reduction in CD19+survivin+splenocytes, reduced AChR-specific antibodies, and retention of AChR at the neuromuscular junction. This result suggests that targeting extracellular survivin directly would be an advantageous therapeutic approach.

In this example, we found the expression of survivin on the cell surface of CD20+ cells suggesting that the survivin may have a role in cell-cell signaling. In the tumor microenvironment, survivin expression in exosomes appears to support cell proliferation and treatment resistance. In the described model, the localization of survivin to the extracellular region would allow for recognition by an antibody and may inhibit survivin's role in supporting persistence of autoreactive cells.

To gain insight to the expression of survivin in the B cells involved in MG, we took advantage of an animal model of MG and used the present antibody to survivin. The study demonstrated the efficacy of reducing survivin by an antibody approach to ameliorate weakness and AChR-specific antibody production in an EAMG mouse model. The ability to target survivin through antibody recognition in the autoreactive cells as described herein can be used as a therapeutic for MG.

Although the present disclosure has been described using specific embodiments and examples, routine modifications will be apparent to those skilled in the art and such modifications are intended to be within the scope of the disclosure.

1. A method for treating survivin-positive autoimmune disease in an individual comprising administering a composition to the individual who has survivin-positive autoimmune disease, said composition comprising an antibody that is specific for survivin.

2. The method of claim 1, wherein the antibody has been generated in response to a peptide which is 9 to 23 amino acids long and comprises the sequence QMFFCF (SEQ ID NO:3).

3. The method of claim 2, wherein the antibody has been generated in response to a peptide which has the sequence DLAQMFFCFKELEGW (SEQ ID NO:4).

4. The method of claim 1, wherein the antibody is a monoclonal antibody.

5. The method of claim 1, wherein the antibody is a chimeric antibody, a humanized antibody, a human antibody, a single chain antibody, a bispecific antibody or a multispecific antibody.

6. The method of claim 1, wherein the antibody has an isotype IgG1, IgG2, IgG3, IgG4, IgM, IgA, IgD or IgE.

7. The method of claim 6, wherein the antibody has an isotype of IgG2b or IgG1.

8. The method of claim 1, wherein the antibody comprises a heavy chain variable region and light chain variable region, wherein,
a) the heavy chain variable region comprises a VH CDR1 comprising the sequence of SEQ ID NO:7, a VH CDR2 comprising the sequence of SEQ ID NO:8, and a VH CDR3 comprising the sequence of SEQ ID NO:9, and the light chain variable region comprises a VL CDR 1 comprising the sequence of SEQ ID NO:10, a VL CDR2 comprising the sequence of SEQ ID NO:11, and a VL CDR3 comprising the sequence of SEQ ID NO: 12; or
b) the heavy chain variable region comprises a VH CDR1 comprising the sequence of SEQ ID NO:13, a VH CDR2 comprising the sequence of SEQ ID NO: 14, and a VH CDR3 comprising the sequence of SEQ ID NO: 15, and the light chain variable region comprises a VL CDR 1 comprising the sequence of SEQ ID NO: 16, a VL CDR2 comprising the sequence of SEQ ID NO:17, and a VL CDR3 comprising the sequence of SEQ ID NO:18.

9. The method of claim 8, wherein the heavy chain variable region comprises an amino acid sequence having at least a 90% sequence identity to SEQ ID NO: 19 and the light chain variable region comprises an amino acid sequence having at least 90% sequence identity to SEQ ID NO:20.

10. The method of claim 8, wherein the heavy chain variable region comprises an amino acid sequence having at least a 90% sequence identity to SEQ ID NO:21 and the light chain variable region comprises an amino acid sequence having at least 90% sequence identity to SEQ ID NO:22.

11. The method of claim 1, wherein the autoimmune disease is myasthenia gravis.

12. The method of claim 1, wherein the autoimmune disease is rheumatoid arthritis.

13. The method of claim 1, wherein the autoimmune disease is graft versus host disease.

14. The method of claim 1, wherein the autoimmune disease is transplant tissue rejection.

15. The method of claim 1, wherein the autoimmune disease is caused by therapy with immune checkpoint inhibitors.

16. The method of claim 1, wherein the autoimmune disease is multiple sclerosis.

17. The method of claim 1, wherein the autoimmune disease is: achalasia, Addison's disease, adult Still's disease, alopecia areata, amyloidosis, ankylosing spondylitis, anti-GBM/Anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, Castleman disease (CD), celiac disease, Chagas disease, chronic inflammatory demyelinating polyneuropathy (CIDP), chronic recurrent multifocal osteomyelitis (CRMO), Churg-Strauss Syndrome (CSS), eosinophilic granulomatosis (EGPA), Cogan's syndrome, cold agglutinin disease, congenital heart block, COVID-19-associated multisystem inflammatory syndrome in children (MIS-C), Coxsackie myocarditis, CREST syndrome, Crohn's disease, dermatitis herpetiformis, dermatomyositis, Devic's disease (neuromyelitis optica), discoid lupus, Dressler's syndrome, endometriosis, epidermolysis bullosa acquisita, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), Herpes gestationis, pemphigoid gestationis (PG), hidradenitis suppurativa (HS), hypophysitis, IgA-mediated bullous dermatosis, IgA nephropathy, IgG4-related sclerosing disease, immune thrombocytopenia purpura (ITP), inclusion body myositis (IBM), inflammatory bowel disease, interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosis, ligneous conjunctivitis, linear IgA disease (LAD), lupus, Lyme disease, Meniere's disease, microscopic polyangiitis (MPA), mixed connective tissue disease (MCTD), Mooren's ulcer, Mucha-Habermann disease, multifocal motor neuropathy (MMN), multiple sclerosis, myositis, narcolepsy, neonatal Lupus, neuromyelitis optica, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II and III, polymyalgia rheumatica, polymyositis, post-myocardial infarction syndrome, post-pericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis/giant cell arteritis, thrombocytopenia purpura (TTP), thyroid eye disease (TED) or Graves' Disease, Tolosa-Hunt syndrome (THS), transverse myelitis, type 1 diabetes mellitus, ulcerative colitis (UC), undifferentiated connective tissue disease (UCTD), uveitis, vasculitis, vitiligo or Vogt-Koyanagi-Harada Disease.

18. The method of claim 8, wherein the autoimmune disease is myasthenia gravis.

19. The method of claim 9, wherein the autoimmune disease is myasthenia gravis.

20. The method of claim 10, wherein the autoimmune disease is myasthenia gravis.
